(19) United States
(12) Patent Application Publication
(10) Pub. No.: US 2015/0044262 A1
(43) Pub. Date: Feb. 12, 2015

(54) ENCAPSULATION OF PERFUMES

(71) Applicant: Givaudan S.A., Vernier (CH)

(72) Inventors: Cédric Geffroy, Poitier (FR); Sophie Sonia Schreiber, Taverny (FR); Marcus James Goodall, Hythe Kent (GB); Addi Fadel, Paris (FR); Ian Michael Harrison, Poissy (FR)

(21) Appl. No.: 14/364,394

(22) PCT Filed: Dec. 21, 2012
(86) PCT No.: PCT/EP2012/076560

§ 371 Date: Jun. 11, 2014

(30) Foreign Application Priority Data
Dec. 22, 2011 (EP) 11290604.5

(51) Int. Cl. A61K 8/11; A61Q 19/10; A61Q 15/00; A61K 8/87

(52) U.S. Cl. CPC A61K 8/11; A61K 8/87; A61Q 19/10; A61Q 15/00; A61K 2800/10; A61K 2800/95; A61K 2800/56; A61K 2800/412; A61K 2800/40
USPC 424/401; 424/65; 510/513; 510/438; 510/130

(57) ABSTRACT

Core-shell capsules suitable for perfuming a consumer product comprising a polymeric shell surrounding and encapsulating a perfume-containing oil core, the mean diameter (D50) of which capsules is about 5 to 250 microns and which capsule is adapted to be ruptured to release perfume contained in the core under a rupture force of less than 2 milli Newtons (mN).

The present invention is concerned with perfume-containing capsules and methods of forming same. The invention is also concerned with consumer products containing said capsules, in particular, consumer products that are used to perfume the human or animal body.

Perfume-containing capsules are known in the art. The capsules may be so-called “core-shell” capsules, which consist of a generally spherical shell that is formed around a core containing the perfume and indeed any other ingredients, which it is desired should be encapsulated. The shell may have a barrier function thereby protecting the perfume from the environment external of the capsule, but it may also act as a means of modulating the release of perfume.

The nature and composition of the shell can influence the manner in which perfume is released from a core-shell capsule. Thus, a shell may be water soluble or water swellable and perfume release may be actuated in response to exposure of the capsules to a moist environment. Similarly, if a shell is temperature sensitive, a capsule might release perfume in response to elevated temperatures. Capsules may also release perfume in response to shear forces applied to the surface of the capsules.

A variety of methods are known for the production of core-shell capsules. One such method is interfacial polymerisation. Interfacial polymerisation typically proceeds with the formation of a fine dispersion of oil droplets (the oil droplets will contain perfume or any other material that is to be encapsulated) in an aqueous continuous phase. The dispersed droplets form the core of the future capsule and the dimensions of the dispersed droplets directly determine the size of the subsequent capsules.

Capsule wall-forming materials (monomers or oligomers) are contained in both the dispersed phase (oil droplets) and the aqueous continuous phase and they react together at the phase interface to build a polymeric wall around the oil droplets thereby to encapsulate the droplets and form core-shell capsules. By means of the appropriate selection of wall-forming materials, one can form cross-links as the polymer wall forms. The extent of cross-linking can affect such factors as the hardness, brittleness, and permeability of the capsule wall.

Interfacial polymerisation offers formulators a convenient and versatile means for encapsulating perfumes as well as other ingredients. This versatile process can be used to form capsules having wide-ranging dimensions. However, relatively small capsules, that is, capsules with mean diameters (D50) ranging between about 1 to 250 microns, more particularly 2 to 50 microns can be more complicated to prepare and perfumes, once encapsulated, can be more prone to leach out of such small capsules, particularly if the capsules are intended to have relatively thin shells.

There remains a need to provide core-shell capsules having relatively small diameters, which are stable during handling and storage, and yet which in use in a consumer product will rupture by compression to release a perfume. There also remains a need for reliable methods of forming such core-shell capsules.

Applicant has now provided core-shell capsules and methods of forming same, which overcome problems in the prior art.

The invention provides in a first aspect a core-shell capsule comprising a polymeric shell surrounding and encapsulating a perfume-containing oil core, the mean diameter (D50) of which capsules is about 1 to 250 microns, more particularly 2 to 50 microns, still more particularly about 3 to about 20 microns and which capsule is adapted to be ruptured to release perfume contained in the core under a rupture force of less than 2 milli Newtons (mN), more particularly less than 1.5 mN, still more particularly less than 1.0 mN, e.g. from 2 mN to 0.025 mN.

The rupture force needed to rupture the capsules can be measured by a technique known in the art as micro-manipulation. The principle of the micro-manipulation technique is to compress single microcapsules between two parallel surfaces. Single microcapsules are compressed and held, compressed and released, and compressed to large deformations or rupture at a pre-set speed. Simultaneously, the force being imposed on them and their deformation can be determined. The technique uses a fine probe, about 10 μm in diameter, positioned perpendicular to the surface of the capsule sample. The probe is connected to a force transducer, which is mounted on a 3-dimensional micro-manipulator that can be programmed to travel at a given speed. The whole process is carried out on an inverted microscope. From the curve of force versus sampling time, the relationship between the force and the microcapsule deformation to bursting, and its initial diameter are obtained.

The technique of micro-manipulation is more fully explained in Zhang, Z., Saunders, R. and Thomas, C. R., Micromanipulation measurements of the bursting strength of single microcapsules, Journal of Microencapsulation 16(1), 117-124 (1999), which document is incorporated herein by reference.

Mean diameter (D50) values are measured by laser diffraction. Laser diffraction methods as well as apparatus for measuring same are well known in the art and warrant no detailed discussion herein.

The invention provides in an embodiment capsules as herein described that have a shell thickness below 0.2 microns. Shell thickness can be determined visually using microscopy, such as scanning electron microscopy.

The invention provides in an embodiment capsules as herein described formed by the formation of a polymeric shell around perfume-containing oil droplets by a process of interfacial polymerisation.

In an embodiment of the present invention polymeric shell may be formed of any material that can be utilised to form a shell by interfacial polymerisation.

In an embodiment of the present invention polymeric shell may be formed of a synthetic polymer.

In an embodiment of the present invention capsule polymeric shell is formed of polyurea, polyamide, hybrid polymers made up of a mixture of organic and inorganic monomers or oligomers, or any other polymer that can be formed around a core by a process of interfacial polymerisation.

Hybrid polymers include those polymers formed from the reaction of isocyanates with appropriately functionalised polysiloxanes, e.g. aminopolysiloxanes, and in particular those hybrid polymers described in US 2011/0118161, which is hereby incorporated by reference in its entirety.

In an embodiment of the present invention polymeric shell material is cross-linked.

The invention provides in an embodiment capsules as herein described, wherein the perfume-containing oil can form an interface with water and the interfacial tension at the oil-water interface is between about 5 and 40 milliNewtons (mN), more particularly 10 to 35 mN, still more particularly 15 to 30 mN.

Whereas it is possible to encapsulate all manner of perfumes and other ingredients in capsules of the present invention, it is possible to prepare small core-shell capsules that are particularly stable in terms of perfume leakage if attention is paid to the perfume-containing oil phase such that the interfacial tension of the interface formed between this oil phase and water falls within the afore-mentioned limits.

It is believed that the interfacial tension that the perfume-containing oil phase exhibits at its interface with water can influence the capsule shell during its formation, and can affect the performance of the capsule in use. Ensuring that the oil phase (at its interface with water) exhibits an interfacial tension in the described range can ensure that the process provides capsules having shells with the requisite strength and rupture properties, water insolubility, lack of porosity, lack of permeability, thickness and hardness that contribute to the stability and performance of the capsules. Capsule shell stability can be a particular problem in the case of capsules having relatively small mean diameters, that is, from about 3 to about 29 microns, or with capsules that in consumer product applications are suspended in liquid bases that contain surfactants or other agents that can compromise the integrity of a capsule shell.

Accordingly, in an embodiment of the present invention there is provided capsules as herein described formed by the formation of a polymeric shell around perfume-containing oil droplets by a process of interfacial polymerisation, the process comprising the step of creating a perfume-containing oil phase that forms an oil-water interface having an interfacial tension with the afore-mentioned limits

The measurement of interfacial tension at liquid-liquid interfaces is well known in the art and doesn't warrant a detailed discussion herein. Interactions between molecules in two liquids of differing densities cause the formation of an interface. To deform this interface requires an input of energy, the work needed for this deformation is known as the interfacial tension. This parameter is similar in principle to surface tension, in which the light liquid phase is replaced with gas.

Interfacial tension measurements were determined by measuring the tension at an oil/water interface according to the Du Noüy ring method. The measurements may be made using a tensiometer, for example a using KRÜSS K100 tensiometer.

The water phase consists of distilled water, in particular distilled water exhibiting a conductivity lower than 80 microS/cm

The skilled person is acquainted with methods of measuring interfacial tension and the apparatus used in such measurements. A tensiometer such as the K100 referred to hereinabove comprises a probe (or ring in the case of the DU Noüy ring method), a precision balance from which the probe is suspended and a motorised sample carrier that provides the required vertical movement. The ring has a known circumference and is made from a platinum-iridium alloy. The balance is capable of registering a force as soon as contact is made with a surface or interface. This force, combined with the ring circumference, supplies the necessary values to calculate the IFT.

During the measurement, the ring begins in the high density phase and then the liquid is lowered so a film of the high density liquid is pulled into the light phase, forming a lamella. As with other tensile measurements, the lamella stretches until a maximum force is reached, the liquid then raises further by a percentage of the maximum force and the cycle repeats.

The interfacial tension is then is calculated using the following equation:

σ=(Fmax−Fv)/(L·cos θ)

wherein:
σ=interfacial tension; Fmax=maximum force; Fv=weight of volume of liquid lifted; L=wetted length, θ=contact angle.

The contact angle decreases as force increases, due to the greater extension, until the maximum force is reached, at which the force vector is parallel to the direction of motion making the contact angle 0°. This gives cos θ a value of 1.

Capsules as defined herein can be used in household and personal care products to impart fragrance thereto.

Accordingly, in another aspect of the invention there is provided the use of a capsule as described herein to perfume a consumer product, in particular a household or personal care product.

In yet another aspect of the invention there is provided a method to confer, enhance, improve or modify the odourant properties of a consumer product, e.g. a household or personal care product, which method comprises adding to said product capsules as hereinabove described.

Capsules of the present invention are rupturable or fracturable under compression. Accordingly, they release fragrance in response to application of a frictional force across the shell surface, such as may be experienced when human skin or a textile such as an item of clothing brushes across a capsules surface.

The recent publication wo2010/049235 discloses an antiperspirant composition containing core-shell capsules that are described as water-insoluble, somewhat brittle and shear-sensitive. Fragrance release occurs primarily by application of frictional forces such as the movement of apparel against the skin. The capsules described in this document are formed of cross-linked gelatin.

However, despite attempts to make fracturable gelatine capsules, they are not clearly rupturable under compression. There is a tendency for fragrance oil contained in the core to partition through the shell reducing the pressure inside the capsules. As such, over a period of time, gelatin capsules tend to behave as a sponge when compressed. Moreover, cross-linked gelatine is partly swellable by water, which leads to the diffusion of perfume on neat and in the presence of moisture over time.

The provision of consumer products, in particular, household and personal care products, containing core-shell capsules as described herein that reliably release their perfume when subjected to shear forces, such as the frictional force of skin against human or animal skin or skin against an inanimate surface such as a textile addresses an unmet need.

Furthermore, by means of the present invention it is possible to encapsulate perfume ingredients in very small capsules, without the capsules being susceptible to substantial leakage.

Small capsules are particularly attractive in certain personal care applications. The applicant surprisingly found that they adhere tenaciously to human skin even after the capsules are exposed to humid conditions such as rinse water or sweat. However, even thought small diameter capsules are desirable for use in humid conditions, nevertheless they are also beneficial across all applications and product types simply because they provide a larger population of capsules for a given mass of encapsulated perfume, which will promote a long-lasting fragrancing effect.

In a particular embodiment of the present invention there is provided a personal care product for fragrancing human or animal skin or hair comprising capsules as hereinabove defined.

In an embodiment of the present invention there is provided a personal care product for fragrancing human or animal skin or hair comprising capsules as hereinabove defined, which is a rinse-off or leave-on product.

In an embodiment of the invention the leave-on product may be a deodorant, for example an under arm deodorant such as a roll-on or stick deodorant or an antiperspirant aerosol spray, or a body lotion, or body spray, or cream, or a hair cream such as a combing cream, or talcum powder.

In an embodiment of the present invention the rinse-off product may be a shower gel, solid or liquid soap, a shampoo or a conditioner.

In an embodiment of the present invention the product contains capsules that have a mean diameter (D50) of 1 to 75 microns, more particularly 2 to 50 microns or 3 to 20 microns or 4 to 15 microns.

In an embodiment of the present invention in a rinse-off product the capsules have a mean diameter (D50) of 5 to 10 microns.

In an embodiment of the invention in a leave-on product that is a body cream or combining cream, the capsules have a mean diameter (D50) of 10 to 15 microns.

In an embodiment of the invention that is a leave-on product that is an under arm deodorant product of the roll-on variety, the capsules have a mean diameter (D50) of 10 to 15 microns.

In an embodiment of the present invention that is a leave-on product of the aerosol deodorant type, the capsules have a mean diameter (D50) of between 10 to 75 microns.

When aerosol compositions are employed the capsule mean diameter (D50) may vary within wide limits. At the lower limit the mean diameter should not be lower than 10 microns because of considerations of lung penetration of fine particles during spraying. The upper limit is controlled by the considerations of the free passage of particles through standard spray nozzles. Currently, it is understood that for conventional nozzles, the mean diameter (D50) should not exceed 75 microns.

The capsules described herein can be employed to encapsulate all manner of perfume ingredients that are useful in consumer products, and in particular personal care products.

In general terms, perfuming ingredients belong to chemical classes as varied as alcohols, ketones, esters, ethers, acetates, nitriles, terpene hydrocarbons, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said ingredients may also be compounds known to release in a controlled manner various types of perfuming compounds.

Consumer products of the present invention, in addition to containing perfumed capsules as described herein, may additionally comprise perfume in unencapsulated form, or perfume encapsulated in other capsules that differ from the capsules of the present invention. For example, consumer products may contain perfumed encapsulates that deliver perfume as a result of exposure to moisture.

Consumer products of the present invention may also comprise all manner of ingredients commonly used in such products other than to provide a pleasant smell. For example, said ingredients might be selected that acts as an aid to processing a product, or it may improve handling or storage. It might also be an ingredient that provides a consumer benefit desirable in such products, such as imparting colour or texture to human skin or hair. It might also be an ingredient that imparts light resistance or chemical stability to one or more ingredients contained in the product. A detailed description of the nature and type of ingredients commonly used in such products cannot be exhaustive, but said ingredients are well known to a person skilled in the art. Examples of ingredients include solvents and co-solvents; surfactants and emulsifiers; viscosity and rheology modifiers; thickening and gelling agents; preservative materials; pigments, dyestuffs and colouring matters; extenders, fillers and reinforcing agents; stabilisers against the detrimental effects of heat and light, bulking agents, buffering agents, antioxidants and the like.

Furthermore, the capsules of the present invention can be used in all the fields of modern perfumery to positively impart or modify the odour of a product into which said capsules are added.

The nature and type of the constituents of a perfumed product do not warrant a more detailed description here, which in any case would not be exhaustive, the skilled person being able to select them on the basis of its general knowledge and according to the nature and the desired effect of said product.

Examples of suitable products include perfumed soaps, shower or bath salts, mousses, oils or gels, hygiene products or hair care products such as shampoos, body-care products, deodorants and antiperspirants.

The proportions in which the capsules can be incorporated into personal care products vary within a wide range of values. These values are dependent on the nature of the product to be perfumed and on the desired olfactive effect. Typically however, products may comprise up to 5% by weight or more of the encapsulated perfume.

A variety of methods are known for the production of core-shell capsules using interfacial polymerisation techniques. Processes typically proceed by the formation of a fine dispersion (conventionally an emulsion) of the perfume-containing oil, in a continuous aqueous phase. The drops of emulsion (or dispersed particles) form the core of the future capsule. The dimensions of the dispersed phase particles directly determine the size of the subsequent capsules. The interfacial tension of the oil phase can be maintained with the above defined range, particularly when it is desirable to produce capsules with small diameters, that is, a D50 in the order of 1 to 50 microns, more particularly 2 to 40 microns, still more particularly 3 to 20 microns.

In a process of interfacial polymerisation monomers or oligomers must react to form the capsule shell. The reactive monomers or oligomers are contained in separate phases and they react at the interface between the continuous and dispersed or discontinuous phase. In this way, as they react with one another at the phase interface, the resultant polymer is already localized at the phase interface. A method of this type can therefore be carried out in a technically simple and reproducible manner.

In a particular embodiment of the present invention the process of forming the core-shell capsules comprises:—

a first step wherein an oil phase is formed containing a perfume to be encapsulated and a monomer or oligomer suitable as a reactant in the formation of the capsule shell;
a second step in which the oil phase is dispersed (e.g. emulsified) in an aqueous continuous phase, wherein the dispersed droplets are substantially of the size of the capsules to be formed;
a third step in which a monomer or oligomer suitable as a reactant for the monomer or oligomer contained in the oil phase is added to the aqueous phase of the dispersion or emulsion to effect an interfacial reaction between the two components leading to the formation of capsule walls; and optionally
a fourth step in which the freshly formed capsules are subjected to subsequent treatment including, e.g. temperature, residence time and/or additional auxiliary materials to harden the capsules.

The monomer or oligomer contained in the oil phase may be a polyfunctional electrophile such as a (poly)isocyanate or a diacyl chloride. The aqueous phase may then contain a polyfunctional nucleophile, such as a polyfunctional amine. If it is intended to have a cross-linked capsule shell, at least one of the components in the dispersed phase or the continuous phase must be at least tri-functional.

Although the third step is described as adding the monomer or oligomer after the dispersion or emulsion is formed, it is also possible that the monomer or oligomer can be added to the aqueous phase prior to dispersion or emulsification.

Conventionally, protective colloids may be added to the aqueous phase, for example polyvinyl alcohol, carboxymethylcellulose, emulsifiers and/or stabilizers. These materials are typically employed to prevent coalescence of the dispersed phase droplets.

In a particular embodiment of the present invention the capsule shell is formed of polyurea polymer. A process for producing polyurea capsules by a process of interfacial polymerisation is provided hereunder, although the skilled person will understand that the general conditions of forming the dispersed oil phase and the subsequent shell-forming conditions may be employed in the preparation of other capsules such as polyamide, melamine, polyacrylic as well as hybrid capsules.

Polyurea capsules can be prepared according to the following general procedure: An aqueous phase may be prepared of water to which a surfactant and/or a protective colloid such as those indicated below have been added. This phase may be stirred vigorously for a time period of only a few seconds up to a few minutes. A hydrophobic phase may then be added. The hydrophobic phase will contain a perfume oil to be encapsulated, and an isocyanate. The hydrophobic phase may also include suitable solvents. After a period of vigorous stirring, an emulsion is obtained. The rate of stirring may be adjusted to influence the size of droplets of hydrophobic phase in the aqueous phase.

An aqueous solution containing an amine reactive towards the isocyanate is then added to affect a polyaddition reaction. The amount of amine which is introduced may be in excess, relative to the stoichiometric amount needed to convert the free isocyanate groups into urea groups.

The polyaddition reaction may take place generally at a temperature ranging from approximately 0 to 100 degrees centigrade for a period of time ranging from a few minutes to several hours.

The skilled person will appreciate that polyamides may be formed in a similar manner by replacing the isocyanate with a suitable co-reactant for the amine such as an acyl chloride.

Conditions for creating capsules by interfacial polyaddition are well known in the art and no further general discussion is needed here. Specific description relating to the preparation of the capsules is provided in the examples below.

Amines useful in the formation of capsules include those compounds containing one or more primary or secondary amine groups which can react with isocyanates or acyl halides to form polyurea or polyamide bonds respectively. When the amine contains only one amino group, the compound will contain one or more additional functional groups that would form a network through a polymerisation reaction.

Examples of suitable amines include 1,2-ethylenediamine, 1,3-diaminopropane, 1,4-diaminobutane, 1,6-diaminohexane, hydrazine, 1,4-diaminocyclohexane and 1,3-diamino-1-methylpropane, diethylenetriamine, triethylenetetramine and bis(2-methylaminoethyl) methylamine.

Other useful amines include poly ethyleneamine (CH2CH2NH)n such as ethyleneamine, diethyleneamine, ethylene diamine, triethylenetetramine, tetraethylenepentamine; poly vinylamine (CH2CHNH2)n sold by BASF (Lupamine different grades); poly ethyleneimine (CH2CH2N)x-(CH2CH2NH)y-(CH2CH2NH2)z sold by BASF under Lupasol grades; poly etheramine (Jeffamine from Huntsman); guanidine, guanidine salt, melamine, hydrazine and urea.

A particularly preferred amine is a polyethyleneimine (PEI), more particularly a PEI from the Lupasol range supplied by BASF, still more particularly Lupasol PR8515.

Isocyanates useful in the formation of polyurea microcapsules include di- and tri-functionalised isocyanates such as 1,6-diisocyanatohexane, 1,5-diisocyanato-2-methylpentane, 1,5-diisocyanato-3-methylpentane, 1,4-diisocyanato-2,3-dimethylbutane, 2-ethyl-1,4-diisocyanatobutane, 1,5-diisocyanatopentane, 1,4-diisocyanatobutane, 1,3-diisocyanatopropane, 1,10-diisocyanatodecane, 1,2-diisocyanatocyclobutane, bis(4-isocyanatocyclohexyl)methane, or 3,3,5-trimethyl-5-isocyanatomethyl-1-isocyanatocyclohexane.

Other useful isocyanates include also the oligomers based on those isocyanate monomers, such as homopolymer of 1,6-diisocyanatohexane. All those monomers and oligomers are sold under the trade name Desmodur by Bayer. Also included are the modified isocyanates and in particular, the water dispersible isocyanate such as Hydrophilic Aliphatic Polyisocyanate based on Hexamethylene Diisocyanate, (sold under the name BAYHYDUR)

Acyl halides useful in the formation of polyamide microcapsules include di- and tri-functionalised acyl halides, commonly acyl chloride, such as linear halides including malonyl halide, glutarhyl halide, adipoyl halide, pimeloyl halide, sebacoyl halide, or such as cyclic halide including phthaloyl, isophthaloyl or terephthaloyl halide, benzene tricarbonyl trichloride.

The classes of protective colloid or emulsifier, which may be employed include maleic-vinyl copolymers such as the copolymers of vinyl ethers with maleic anhydride or acid, sodium lignosulfonates, maleic anhydride/styrene copolymers, ethylene/maleic anhydride copolymers, and copolymers of propylene oxide, ethylenediamine and ethylene oxide, polyvinylpyrrolidone, polyvinyl alcohols, fatty acid esters of polyoxyethylenated sorbitol and sodium dodecylsulfate.

Suitable solvents include aliphatic hydrocarbons, chlorinated aliphatic hydrocarbons, alicyclic hydrocarbons, chlorinated alicyclic hydrocarbons, and aromatic or chlorinated aromatic hydrocarbons. More particularly, solvents include cyclohexane, octadecane, tetrachloroethylene, carbon tetrachloride, xylenes, toluene, chlorobenzene and alkylnaphthalenes.

The embodiments of the invention described herein above may be read alone or they may be read together in any combination to form specific embodiments of the invention.

In order to further illustrate the present invention and the advantages thereof, the following specific examples are given, it being understood that same are intended only as illustrative and in no way limitative.

### EXAMPLE 1

### Preparation of Polyurea Capsules

An oil phase was prepared when Desmodur W (Bayer) and Bayhydur XP2547 (Bayer) were added in perfume oil at a level of 12.6% and 3.4% respectively.

An aqueous phase (Solution S1) was prepared by adding Luviskol k90 (BASF) to water, at a level of 4.5%. The pH of the solution was adjusted at in by addition of a buffer pH=10 at 0.5%.

An aqueous phase (Solution S2) was prepared by adding Lupasol PR8515 (BASF) to water, at a level of 20%.

Capsules were prepared according to the following procedure:

300 g of the oil phase was mixed with 600 g of solution S1, to form an oil-in-water emulsion, in a 1 L reactor equipped with a MIG stirrer operating at 1000 rpm. After 30 minutes of mixing, wog of solution S2 was added over a period of 1 minute. After 30 minutes, the slurry was heated up to 70° C. (1H), then kept for 2H at 70° C., then heated to 80° C. and kept for 1 H at 80° C., then heated to 85° C. and kept for 1 H at 85° C., then cooled to 70° C. and kept for 1 H at 70° C. before final cooling at 25° C.

### EXAMPLE 2

Perfumes A through I were encapsulated in polyurea capsules formed according to the general method of Example 1. The capsules are intended for roll-on deodorant applications.

| | Encapsulated | Measured | Mean particle size | Solid |
|---|---|---|---|---|
| Capsule | oil | IFT | (d50, μm) | content (%) |
| 1 | Perfume A | 46 | 43 | 34.3 |
| 2 | Perfume B | 30 | 12 | 37.8 |
| 3 | Perfume C | 23 | 6 | 37.2 |
| 4 | Perfume D | 12 | 15 | 28.3 |
| 5 | Perfume E | 35 | 36 | 35.8 |
| 6 | Perfume F | 19 | 7 | 36.9 |
| 7 | Perfume G | 25 | 5 | 37.3 |
| 8 | Perfume H | 31 | 21 | 36.5 |
| 9 | Perfume I | 28 | 8 | 37.8 |

Interfacial tension measurements were made according to the methodology described hereinabove.

The particle size distribution is measured using the technique of laser diffraction, using a Mastersizer 2000 supplied by Malvern. The technique is based on the principle that the light from a coherent source, in this case the laser beam, will scatter as particles pass through the beam, with the angle of the scattered light being directly related to the size of the particles. A decrease in particle size results in a logarithmic increase in the observed scattering angle. The observed scattering intensity is also dependent on particle size and diminishes relative to the particle's cross-sectional area. Large particles therefore scatter light at narrow angles with high intensity, whereas small particles scatter at wider angles but with low intensity. Detectors are used to measure the scattered light pattern produced over a wide range of angles and, hence, determine the particle size distribution of the sample using an appropriate optical model.

For the measurement of the particle size, the sample was placed in the Malvern Hydro2000 SM module, supplied with the Mastersizer 2000, for the measurement of wet dispersions. The supplied software was used to transform the measured scattered light pattern into the particle size distribution. The optical model parameters used were 1.47 and 0 for the refractive index and absorption index, respectively. Sample measurement was taken over a period of five seconds using 5000 measurement snaps.

The efficiency of perfume encapsulation is determined by measuring the solid content or dry weight of the capsule dispersion. To this end, an infra-red balance is used. Such a balance is the Moisture Analyzer HR83 as supplied by Mettler-Toledo. Approximately 2 g of the capsule dispersion is placed on the balance by use of a suitable cellulose or fibreglass support, such as that supplied by Mettler-Toledo. The capsule dispersion is heated at a temperature of 120° C. until dry, as indicated by the balance by means of a constant and unchanging weight. Since the intended use of this particular balance is to give a measure of moisture, the measurement indicates the level of water lost from the capsule dispersion and, hence, the solid content or dry weight. The theoretical solid content is 37.4%. Values for solid content of the various encapsulated oils are given in the table, below.

Solids content analysis is a measure of the material remaining after evaporation of volatiles. It provides an assessment of shell integrity (porosity) and the ability to retain perfume under stress conditions of temperature. As such, it is an indication of leakage and stability over time. For the capsules of Example 2 the solids content was anticipated to be around 37.4% (approximately 25 parts perfume and 12 parts capsule). Accordingly, the capsules 1, 4 and 5 performed poorly in the sense that more than 10% of the expected quantity of encapsulated perfume was lost.

### EXAMPLE 3

A panel testing of 20 subjects was used to validate performance of 1% dispersion of Capsule 9 [IFT value 28; Particle size 8 microns] and Capsule 4 [IFT value 12; Particle size 15 microns] in a roll-on water-based deodorant application.

Performance was assessed by the panel on neat (perception by consumer upon opening sample and before application), 1 hour after application, 5 hours after application. The 10 hour measurement was made before and after activation (rubbing), and at 24 hours after shower also upon rubbing.

The results are shown summarized below:

| | | | | | 24 hours after |
|---|---|---|---|---|---|
| | | | | 10 hours | shower |
| Intensity perceived | Neat | 1 h | 5 h | (before/after) | (before/after) |
| Capsule 9 (containing | 7 | 6 | 4 | 2/3 | 1/2 |
| Perfume I) | | | | | |
| Free perfume I | 7 | 6 | 3 | 1/1 | 0/0 |
| Capsule 4 (containing | 7 | 6 | 4 | 1/2 | 0/0 |
| Perfume D) | | | | | |
| Free perfume D | 7 | 6 | 3 | 1/1 | 0/0 |

A 10 point intensity scale was used to assess the intensity of the perfume performance for both cases. The formulations containing the encapsulation 9 showed superior performance as illustrated above with significance above 95%. In particular, it should be noted that the capsules remained on skin even after shower.

### EXAMPLE 4

The procedure below describes the washing and evaluation methods used to measure the performance of capsule technologies in shower gel products under controlled laboratory conditions and in a home use test (HUT).

Sample Preparation

The capsule sample was added to the base and stirred using a mechanical stirrer which has a configuration that generates movement of the mixture from the bottom to the top. A propeller stirrer or angled turbine stirrer is preferred.

Shower Gel Bases

A Givaudan standard Shower Gel base (DBA002) was utilized for these assessments.

| INGREDIENTS | SUPPLIER | INCI NAME | % W/W |
|---|---|---|---|
| PHASE A | | | |
| TEXAPON N 40 | COGNIS | Sodium laureth sulfate | 38.00 |
| DEHYTON K | HENKEL | Cocamidopropylbetaine | 8.00 |
| EUPERLAN | SIDOBRE | Glycol distearate & laureth 4 | 5.00 |
| PK 3000 | SINNOVA | & Cocamidopropylbetaïne | |
| DEIONISED | | Water | qsp 100 |
| WATER | | | |
| PHASE B | | | |
| MERQUAT S | SCHMITT- | Polyquaternium-7 | 0.40 |
| | JOURDAN | | |
| NIPAGUARD | NIPA | DMDM Hydantoin | 0.50 |
| DMDMH | | | |
| PANTHENOL | ROCHE | Panthenol | 2.00 |
| 75 l | | | |
| PHASE C | | | |
| SODIUM | PROLABO | Sodium Chloride | 1.20 |
| CHLORIDE | | | |
| TRILON B | BASF | Tetrasodium EDTA | 0.25 |
| DEIONISED | | Water | 10.00 |
| WATER | | | |
| PERFUME | GIVAUDAN | Fragrance | 1.50 |

pH = 5.5 to 6.5
% surfactants active material = 15.87%

Process:

Mix Phase A except water with stirring until homogeneous. Add water in two parts. Add constituents of phase B. Add ingredients of phase C previously dissolve in water. Adjust pH to 5.5 at 6

Washing Methodology (Controlled Laboratory Conditions)

Each volunteer washed and dried their forearms with unfragranced shower gel before the trial. Each volunteer would typically have one forearm treated with the control sample, the other with a test/capsule sample. Routinely the sample was applied to the left forearm first. The volunteer would wet the forearm under running water (constant flow and temperature defined by volunteer). A syringe was used to apply 2 ml of product to the outer part of the left forearm. The volunteer, using their free hand, rubbed the product into the arm four times, following a circular motion, up and down the length of the forearm. At this point the volunteer would extend their forearm to be assessed by a group of at least four evaluators. This would be documented as the bloom in-use.

The forearm was then re-wetted under the running water and the volunteer would rub their forearm a further four times. Finally, the forearm was held under running water (for a period of time defined by the volunteer) to allow any foam and residue product to be removed. The volunteer then used a clean terry-toweling flannel to pat dry the area. The arm was, once again extended and assessed for the initial dry skin performance.

The procedure was then repeated for the right arm. Once the initial assessment was complete the volunteers were free to go about their daily business. After 5 hours the volunteers were re-evaluated, before and after rubbing the forearm. The rubbing step was achieved by using a clean terry-towelling flannel and gently rubbing the forearms, four times, in an up down motion.

Washing Methodology (HUT)

A minimum of ten volunteers were required for the trial. Each volunteer was supplied with a 30 g sample of shower gel to take home and a questionnaire to complete. The volunteer would use the shower gel sample in their normal washing routine, in place of their usual products. The volunteer would sell assess their outer forearm at various time points typically, initial, 30 minutes, 1 hour, 2 hours, 4 hours and 6 hours. After the 6 hour assessment the forearm would be gently rubbed with a clean terry-towelling flannel (provided) four times in an up down motion, before a further sell assessment (6 hours after rubbing). The volunteer may also be asked to assess at further time points of 12 and 24 hours as required.

Evaluation of Skin

The performance of the product was evaluated by a panel of assessors, experienced and trained in such evaluations. Each assessor scores the performance on an individual basis and then the results are collated, averaged and analysed for statistical significance (Confidence interval of 95% (Tukey HSD)).

A standard 0-10 scoring system was used, where:

0—No odour

2—Odour is barely perceivable

4—Weak fragrance but perceivable

6—Easily perceivable

8—Strong

10—Very strong

Fragrance intensity Score

| Particle Size | | | | | 5 Hrs |
|---|---|---|---|---|---|
| D(50) | Sample | Bloom | Initial | 5 Hrs | Rub |
| 19 μm | Capsule 1 | 5.9 | 2.4 | 1.9 | 2.9 |
| 14 μm | Capsule 2 | 5.7 | 2.3 | 1.9 | 2.9 |
| 5 μm | Capsule 3 | 5.7 | 3.2 | 1.9 | 4.6* |
| 8 μm | Capsule 4 | 5.7 | 3.4 | 2.0 | 4.8* |

*Performance benefit (Significant p < 0.05).

1. Core-shell capsules comprising a polymeric shell surrounding and encapsulating a perfume-containing oil core, the mean diameter (D50) of which capsules is about 5 to 250 microns and which capsule is adapted to be ruptured to release perfume contained in the core under a rupture force of less than 2 milli Newtons (mN).

2. The capsules according to claim 1 wherein the perfume-containing oil can form an interface with water and the interfacial tension at the oil-water interface is between about 5 and 40 milliNewtons (mN).

3. The capsules according to claim 1 formed by the formation of a polymeric shell around perfume-containing oil droplets by a process of interfacial polymerisation.

4. The capsules according to claim 1 wherein the polymeric shell is formed of a synthetic polymer.

5. The capsules according to claim 1 wherein the polymeric shell is formed of polyurea, polyamide, or hybrid polymers formed from a mixture of organic and inorganic monomers or oligomers.

6. The capsules according to claim 1 wherein the polymeric shell is cross-linked.

7. A process of utilizing capsules as defined in claim 1 to perfume a consumer product, optionally a household product or personal care product.

8. A method to confer, enhance, improve or modify the odourant properties of a consumer product, optionally a household product or personal care product, which method comprises adding to said consumer product capsules as defined in claim 1.

9. A consumer product for fragrancing human or animal skin or hair comprising capsules as defined in claim 1.

10. The consumer product according to claim 9, which is a rinse-off product or a leave-on product.

11. The consumer product according to claim 9, which is a deodorant, an under arm deodorant, a roll-on deodorant a stick deodorant, an antiperspirant aerosol spray, a body lotion, body spray, cream, a hair cream, a combing cream, or talcum powder.

12. The consumer product according to claim 9, which is a shower gel, solid soap, er liquid soap, a shampoo or a conditioner.

13. The consumer product according to claim 9, wherein the capsules have a mean diameter (D50) of 2 to 75 microns.

14. The consumer product according to claim 9 that is a rinse-off product and the capsules have a mean diameter (D50) of 5 to 10 microns.

15. The consumer product according to claim 9, which is a leave-on product that is selected from a body cream or combing cream, wherein the capsules have a mean diameter (D50) of 10 to 15 microns.

16. The consumer product according to claim 9, which is a leave-on product that is selected from an under arm deodorant product of the roll-on variety, and wherein the capsules have a mean diameter (D50) of 10 to 15 microns.

17. The consumer product according to claim 9, which is a leave-on product that is an aerosol deodorant, and wherein the capsules have a mean diameter (D50) of between 10 to 75 microns.

18. A process of forming capsules defined in claim 1, comprising the step of forming a polymeric shell around a perfume-containing oil droplets by a process of interfacial polymerisation.

19. The process according to claim 18 wherein the perfume-containing oil is selected on the basis that it can form an interface with water and the interfacial tension at the oil-water interface is between about 5 and 35 milliNewtons (mN).

20. The process according to claim 18 comprising:—
a first step wherein an oil phase is formed that contains a perfume to be encapsulated and a monomer or oligomer suitable as a reactant in the formation of a capsule shell by interfacial polymerisation;
a second step in which the oil phase is dispersed (or emulsified in an aqueous continuous phase, wherein the dispersed or emulsified droplets are substantially of the size of the capsules to be formed;
a third step in which a monomer or oligomer suitable as a reactant for the monomer or oligomer contained in the oil phase is added to the aqueous continuous phase of the dispersion or emulsion to effect an interfacial reaction between the two components leading to the formation of capsule shells around the dispersed oil phase; and optionally
a fourth step in which the formed capsules are subjected to subsequent treatment including, optionally temperature, residence time and/or additional auxiliary materials to harden the capsules.
